(19) United States
(12) Patent Application Publication
(10) Pub. No.: US 2007/0276304 A1
(43) Pub. Date: Nov. 29, 2007

(54) Apparatus for preventing or minimizing tendinitis

(76) Inventors: Mark Greenberg, Stuart, FL (US); Aldo Burga, Port St. Lucie, FL (US)

Correspondence Address: GOLD & RIZVI, P.A., 600 N. PINE ISLAND ROAD, SUITE 450, PLANTATION, FL, 33324-1311, US

(21) Appl. No.: 11/431,258

(22) Filed: May 10, 2006

(51) Int. Cl. A61F 5/00

(52) U.S. Cl. 602/21

(57) ABSTRACT

An apparatus and method for preventing the development or exacerbation of tendinitis is provided. The apparatus includes a first pad made of a resilient material that is configured to be carried by a digit of the user. The first pad has a palmar face and an oppositely disposed dorsal face that define a thickness therebetween. The thickness of the first pad is selected in order to prevent the digit of the user from fully bending so that impact forces transmitted through the digit are dampened as the muscle pulling on the digit is not completely tight.

### FIELD OF THE INVENTION

The present invention relates generally to an apparatus that is used to prevent the development or exacerbation of tendinitis. More particularly, the present application involves an apparatus configured for positioning on the finger of a user that absorbs impact forces and minimizes flexing of the finger in order to reduce resulting shock imparted to a tendon of the user.

### BACKGROUND OF THE INVENTION

Tendinitis is inflammation or irritation of a tendon and is normally caused through overuse of the tendon and muscles connected thereto. Athletic activities such as tennis, golf, fishing, bicycling, baseball and softball may lead to the development of tendinitis as these place strain on areas prone to this condition such as shoulders, elbows and knees. Although associated with athletic activities, tendinitis may also occur as a result of housecleaning, operating a chainsaw, playing a musical instrument, and through work related activities such as performing repetitive movements while working on an assembly line.

When a user grips an object, such as a tennis racket or a golf club, forces imparted onto the object are transferred through the fingers of the user and along a tendon-muscle-tendon unit responsible for keeping the fingers gripped to the object. Transfer of this impact force results in microscopic tears to the tendon which results in weakening of the tendon and swelling. Various techniques designed to prevent tendinitis have been proposed. One method of preventing tendinitis involves using a brace to press the tendon near the elbow into the muscle mass in order to blunt forces imparted onto the tendon. Braces of this type sometimes employ a shock absorbent gel or other material to absorb forces that are transmitted into the tendon. Although these braces enjoy some degree of success, they are sometimes cumbersome and limit movement. Braces are also problematic in that they require the user to precisely place the brace over the correct area of the arm. Additionally, the user must regularly check the brace and adjust its tightness in order to ensure, proper effectiveness. Readjustment of the brace is sometimes needed, as the forearm of the user tends to enlarge during use. Additionally, the brace may act to cut off blood flow to the forearm and surrounding area if it is improperly set or becomes too tight through use.

Another prior device that was used to address the problem of tendinitis utilized an elastic pull system that acted to dorsiflex the wrist of the user. This device sought to decrease flexion of the wrist when an impact force was applied thereto. This device was problematic in that it was not capable of decreasing full flexion of various digits of the hand of the user and hence did not result in reducing force imparted onto the tendon of the user during impact.

Other solutions to the prevention or reduction of tendinitis involve introducing shock-absorbing properties into the object that is held by the user. For instance, some tennis rackets are designed with shock absorbing strings that are said to reduce shock to the arm of the user. Alternatively, padded handgrips on tennis rackets have been promoted as being beneficial to the reduction of tendinitis.

Other devices have been proposed, for the prevention of tendinitis in the elbow, which interact with the hand of the user. One such device utilizes a rigid ring that is attached to the palmar side of the little finger. The rigid ring prevents the little finger from contacting the object grasped by the user and keeps the little finger in an extended position to avoid overstressing muscles and tendons. However, as the other fingers of the hand then grasp the object, forces normally carried by the little finger are distributed to the other fingers resulting in a greater amount of force applied to the tendons therefrom.

Additional devices have been proposed that are worn on the fingers of the user or are incorporated into a glove that is worn by a user. Once such device is a golf glove that has a resilient rectangular shaped member attached thereon that acts to fill in natural gaps created between the golf club hand grip and the golfer's hand in order to achieve a more positive fit. Although such a glove may work to achieve a better grip, tendinitis is not alleviated as forces imparted onto the golf club are more easily transferred through the glove due to its improved grip.

Another device is known that consists of a sheath that fits around one or more fingers of the user. The sheath has projections that act to cushion the finger and help to achieve a desired gripping effect. The sheath is used to protect the selected areas of the user's hand from cuts, sores and blisters while at the same time permitting maximum movement, tactile sensation and ventilation of the finger. This design does not address the problem of tendinitis. As such, there remains room for variation and improvement within the art.

### SUMMARY

Various features and advantages of the invention will be set forth in part in the following description, or may be obvious from the description, or may be learned from practice of the invention.

The present invention provides for an apparatus and method for preventing the development or exacerbation of tendinitis. The apparatus includes one or more pads that act to prevent the user from grasping an object so that the tendon-muscle-tendon unit in communication with the finger is not completely tight. Forces imparted onto the object are thus slowed and dampened before reaching the elbow of the user. The pads of the apparatus may also be resilient in order to absorb forces imparted onto the object.

In accordance with one exemplary embodiment of the present invention, the apparatus includes a first pad made of a resilient material that is configured for being carried by a digit of the user. The first pad has a palmar face and an oppositely disposed dorsal face. The first pad has a first end and an oppositely disposed second end. At least one of the first and second ends has a concave shape to allow for some degree of bending of the digit of the user.

The present invention also provides for an apparatus as immediately discussed in which the palmar face of the first pad has a concave shape that is configured for receiving a phalange of the user.

Also provided for in accordance with the present invention is an apparatus as previously discussed in which both the first and second ends have a concave shape.

Another exemplary embodiment exists in an apparatus as discussed that further has a second pad made of a resilient material and is configured for being carried by a digit of the user. The second pad has a palmar face and an oppositely disposed dorsal face. The second pad has a first end and an oppositely disposed second end. At least one of the first and second ends has a concave shape to allow for some degree of bending of a digit of the user. Additionally, a third pad may also be incorporated into the apparatus. Like the second pad, the third pad is also resilient and is configured for being carried by a digit of the user. The third pad has a palmar face and an oppositely disposed dorsal face. The third pad has a first end and an oppositely disposed second end in which at least one of the ends has a concave shape in order to allow for some degree of bending of the digit of the user.

Also provided for in accordance with another exemplary embodiment of the present invention is an apparatus for preventing the development or exacerbation of tendinitis. The apparatus includes a first pad made of a resilient material and configured for being carried by a digit of the user. The first pad has a palmar face and an oppositely disposed dorsal face that define a thickness therebetween. The thickness of the first pad is selected so as to prevent the digit of the user from fully bending.

In accordance with another exemplary embodiment, an apparatus is provided as immediately discussed in which the thickness of the first pad is at least nine millimeters.

Also provided for is an apparatus as discussed above in which the palmar face of the first pad has a concave shape that is configured for receiving a phalange of the user. The first pad has a first end and an oppositely disposed second end. At least one of the first and second ends has a concave shape.

In accordance with yet another exemplary embodiment, the apparatus as discussed is provided so that the first pad is a ring that has a uniform width and thickness.

Also provided for in accordance with the present invention is an apparatus as discussed above that includes a second pad made of a resilient material that is configured for being carried by a digit of the user. The second pad has a palmar face and an oppositely disposed dorsal face that define a thickness therebetween. The thickness of the second pad is selected so as to prevent the digit of the user from fully bending. An additional embodiment is disclosed as immediately discussed that includes a third pad made of a resilient material configured for being carried by a digit of the user. The third pad has a palmar face and an oppositely disposed dorsal face that define a thickness therebetween. The thickness of the third pad is selected so as to prevent the digit of the user from fully bending.

The present invention additionally provides for another exemplary embodiment of an apparatus as discussed above that further includes a retaining member configured for retaining the first pad to a digit of the user. Various exemplary embodiments exist in which the retaining member may be a hook and loop type fastener, gauze, athletic tape, an elastic member, a finger cot and/or a glove.

The present invention provides for a method of preventing the development or exacerbation of tendinitis that includes the step of providing a first pad made of a resilient material configured for being carried by a digit of the user. The first pad has a palmar face and an oppositely disposed dorsal face that define a thickness of the first pad therebetween. The thickness of the first pad is selected so as to prevent the digit of the user from fully bending. The method also includes the step of retaining the first pad onto the digit of the user so that the first pad is positioned on the palmar side of the digit.

Another exemplary embodiment of the present invention exists in a method as immediately discussed that further includes the step of grasping an object with a hand onto which the first pad is retained. The object is grasped so that the digit onto which the first pad is retained curls onto the object without tightly grasping the object. The method also includes the step of absorbing impact forces imparted onto the object by way of resiliency of the first pad and by way of slackness of a muscle pulling on the digit.

These and other features, aspects and advantages of the present invention will become better understood with reference to the following description and appended claims. The accompanying drawings, which are incorporated in and constitute part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended Figs. in which:

FIG. 1 is a perspective view of an apparatus located an a finger of the user in accordance with one exemplary embodiment of the present invention.

FIG. 2A is a perspective view of the first pad of FIG. 1.

FIG. 2B is a top view of the first pad of FIG. 1.

FIG. 2C is a front view of the first pad of FIG. 1.

FIG. 2D is a side view of the first pad of FIG. 1.

FIG. 3 is a perspective view of the apparatus of FIG. 1 when the user of the apparatus grasps an object.

FIG. 4A is a perspective view of an apparatus located on a finger of the user in accordance with another exemplary embodiment of the present invention in which the pads of the apparatus are configured as rings.

FIG. 4B is a perspective view of the first pad of FIG. 4A.

FIG. 5 is a perspective view of an apparatus located on a finger of the user in accordance with another exemplary embodiment of the present invention in which the pads are incorporated into a sheath.

FIG. 6 is a perspective view of an apparatus located on a finger of the user in accordance with another exemplary embodiment of the present invention in which the pads are incorporated into a glove.

Repeat use of reference characters in the present specification and drawings is intended to represent the same or analogous features or elements of the invention.

### DETAILED DESCRIPTION OF REPRESENTATIVE EMBODIMENTS

Reference will now be made in detail to embodiments of the invention, one or more examples of which are illustrated in the drawings. Each example is provided by way of explanation of the invention, and not meant as a limitation of the invention. For example, features illustrated or described as part of one embodiment can be used with another embodiment to yield still a third embodiment. It is intended that the present invention include these and other modifications and variations.

It is to be understood that the ranges mentioned herein include all ranges located within the prescribed range. As such, all ranges mentioned herein include all sub-ranges included in the mentioned ranges. For instance, a range from 100-200 also includes ranges from 110-150, 170-190, and 153-162. Further, all limits mentioned herein include all other limits included in the mentioned limits. For instance, a limit of up to about 7 also includes a limit of up to about 5, up to about 3, and up to about 4.5.

The present invention provides for an apparatus 10 that is used to prevent the development or exacerbation of tendinitis. The apparatus 10 includes one or more pads 12, 14 and 16 that are retained on a finger 24 of a user 18. The pads 12, 14 and 16 prevent the user 18 from tightly closing his or her finger 24 around an object 86 so that impact forces applied to the object 86 are dampened upon being transferred through the tendon-muscle-tendon unit of the user 18. Additionally, the pads 12, 14 and 16 are made of a resilient material and sized so as to absorb impact forces applied to the object 86 to further reduce resultant stress. The apparatus 10 may be arranged in a variety of manners in accordance with various exemplary embodiments of the present invention.

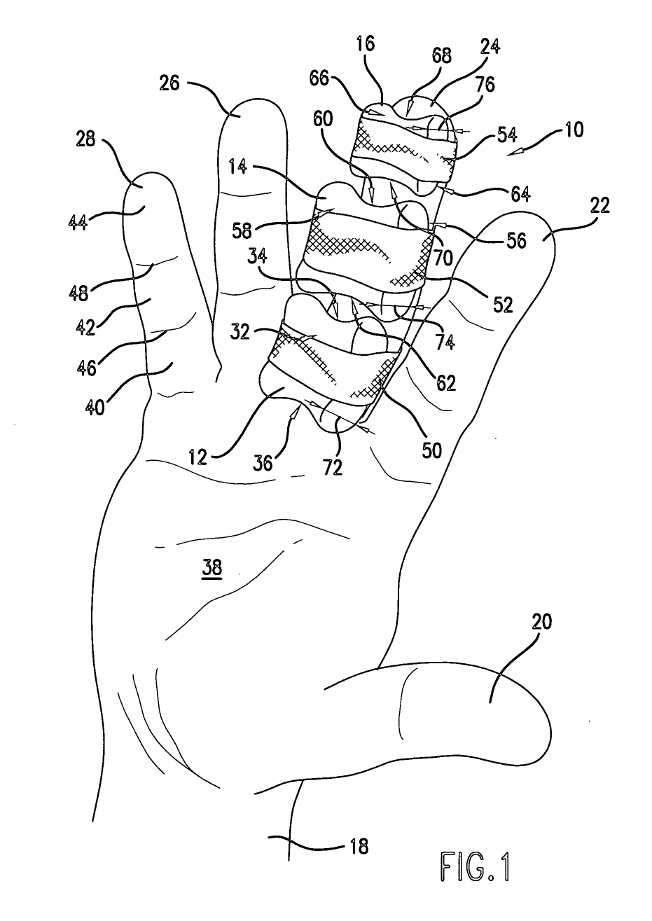

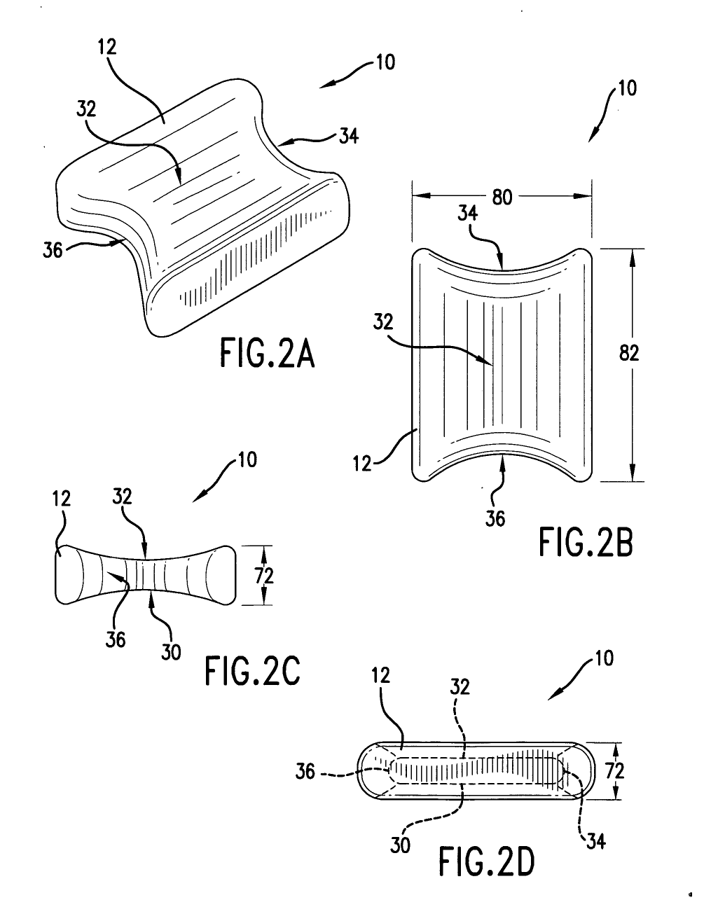

FIG. 1 shows one embodiment of the apparatus 10 retained on the middle finger 24 of the user 18. The apparatus 10 includes first, second and third pads 12, 14 and 16 that are retained onto the middle finger 24 by retaining members 50, 52 and 54. The pads 12, 14 and 16 are positioned on the palmar side of the hand of the user 18. First pad 12 is shown in greater detail in FIGS. 2A-2D. First pad 12 has a palmar face 30 that is configured for contacting the finger 24 of the user 18 and an oppositely disposed dorsal face 32. In accordance with one embodiment, the first pad 12 has a length 82 of nineteen millimeters and a width 80 of fourteen millimeters such that the palmar face 30 and the dorsal face 32 have an area of approximately two hundred and sixty six square millimeters. It is to be understood, however, that the width 80 and length 82 of the first pad 12 may be different in other embodiments. For example, the width 80 may be up to twenty-five millimeters and the length 82 may be up to thirty millimeters in accordance with various exemplary embodiments.

Preferably, the first pad 12 has a thickness 72 of approximately nine millimeters and is made of a soft, shock absorbent material. Materials that may be used to construct the first pad 12 include polyurethane, gel, silicone or foam. Although described as having a preferred thickness 72 of approximately nine millimeters, the thickness 72 of first pad 12 may be up to thirty millimeters in accordance with other exemplary embodiments. Furthermore, the first pad 12 may have a thickness 72 of less than nine millimeters. In fact, experimentation has shown that virtually any thickness will function adequately for the intended purpose as long as there is sufficient compensatory padding around the racket handle. The first pad 12 may be a single, integral piece or may be made of one or more components. For example, the first pad 12 may include a foam section attached to a gel pad that is designed to fit against the finger 24 of the user 18. The first pad 12 may have absorbent properties that act to remove perspiration from the skin of the user 18 during use of the apparatus 10.

As shown in FIG. 1, the first pad 12, second pad 14 and third pad 16 are retained on the middle finger 24 and do not contact one another when the middle finger is not flexed. Although shown as being located on the middle finger 24, it is to be understood that the apparatus 10 may additionally or alternatively be used on the thumb 20, index finger 22, ring finger 26 and/or little finger 28 in accordance with other embodiments. Further, instead of three pads 12, 14 and 16, any number of pads may be employed. For example, in an alternative embodiment one pad 12 is retained on the middle finger 24 while a single pad 14 is retained on the ring finger 26. Selection and location of the pads may be made based upon a particular form of tendinitis affecting the user 18. When the user 18 is experiencing a form of tendinitis commonly known as tennis elbow, the pads may be located on the third finger 26. When the user 18 experiences a form of tendinitis commonly referred to as golfer's elbow, the pads may be located on the index finger 24. If the user 18 experiences both of these forms of tendinitis, the pads may be placed on both the index and middle fingers 24 and 26. As such, various combinations and numbers of pads are envisioned as making up the apparatus 10 in accordance with various embodiments of the present invention.

The little finger 28 is shown in FIG. 1 as having a proximal phalange 40 that is adjacent the palm 38 of the user 18. A proximal interphalangeal joint 46 is located intermediate the proximal phalange 40 and a middle phalange 42 of the little finger 28. A distal interphalangeal joint 48 is located intermediate the middle phalange 42 and a distal phalange 44 of the little finger 28. The aforementioned designations of the little finger 28 is representative of the designations that may be used to describe the index finger 22, middle finger 24 and ring fingers 26. The designations are also representative of the thumb 20, although the thumb 20 lacks both a middle phalange 42 and a distal interphalangeal joint 48.

The first pad 12 is retained on the middle finger 24 in such a manner as to be disposed over the proximal phalange 40 on the palmar side of the hand of user 18. The second pad 14 is disposed over the middle phalange 42 of the middle finger 24 while the third pad 16 is disposed over the distal phalange 44. As with the first pad 12, the second and third pads 14 and 16 are on the palmar side of the hand of the user 18 and are not on the opposite side of the middle finger 24 from the palmar side. In the configuration shown in FIG. 1, the first pad 12 is spaced from the second pad 14, and the second pad 14 is spaced from the third pad 16.

Referring again to FIGS. 2A-2D, the palmar face 30 of the first pad 12 is concave in shape in order to conform to the shape of the proximal phalange 40. The palmar faces 56 and 64 of the second and third pads 14 and 16 may likewise have a concave shape in order to receive the middle and distal phalanges 42 and 44. The dorsal face 32 of the first pad 12 also has a concave shape, although it is to be understood that in other exemplary embodiments that the dorsal face 32 need not be concave in shape. Likewise the second and third pads 14 and 16 may have a dorsal face 58 and 66 that are or are not concave in shape. The first pad 12 has a first end 34 and an oppositely disposed second end 36. The first and second ends 34 and 36 are oriented so as to be on opposite ends of the first pad 12 in the direction of the length 82 of the first pad 12. As shown, both the first and second ends 34 and 36 of the first pad 12 have a concave shape. The ends 34 and 36 are concave in shape in order to accommodate flexing of the middle finger 24 when the user 18 grasps an object 86. The second pad 14 has a first end 60 and a second end 62 that are concave in shape, and the third pad 16 is provided with a first end 68 and a second end 70 that are also concave. Although described as having portions that are concave in shape, it is to be understood that the pads 12, 14 and 16 may be variously shaped in accordance with other exemplary embodiments. For example, the pads 12, 14 and 16 may have normally flat surfaces and be generally rectangular in shape in accordance with certain embodiments.

As shown in FIG. 1, the retaining member 50 is athletic tape that is wrapped around the first pad 12 and contacts the dorsal face 32. The retaining member 50 acts to keep the first pad 12 relatively tightly attached to the middle finger 24 but not so tight as to cut off circulation. The retaining member 50 also prevents the first pad 12 from sliding off of or moving around the middle finger 24 to be positioned on the side of the middle finger 24 opposite the palmar side. Although shown as being athletic tape, the retaining member 50 can be any device capable of retaining the first pad 12 and may be variously configured in other embodiments. For example, the retaining member 50 may be a hook and loop type fastener that wraps around the middle finger 24 and engages the dorsal face 32 of the first pad 12. The retaining member 50 may also be provided as gauze or an elastic member to hold the first pad 12 to the middle finger 24 in other embodiments. Retaining members 52 and 54 may be configured as previously discussed with respect to the first pad 12. The retaining members 50, 52 and 54 may be the same as one another or different in accordance with various exemplary embodiments. The retaining members 20, 52 and 54 may be selected and provided so that readjustment of the pads 12, 14 and 16 of the apparatus 10 is either not, or only infrequently necessary.

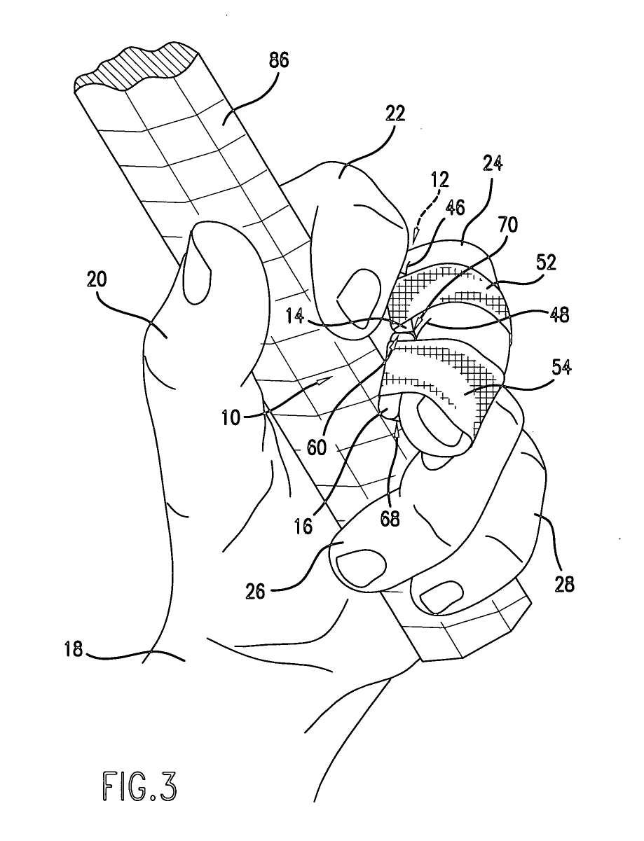

FIG. 3 is a perspective view of the apparatus 10 of FIG. 1 when the hand of the user 18 is bent into a fist in order to grasp an object 86 such as the handle of a tennis racket. During bending of the fingers 20, 22, 24, 26 and 28, the pads 12, 14 and 16 may touch one another but should desirably not pinch the skin of the user 18. The pads 12, 14 and 16, function to keep the middle finger 24 from directly touching the grip surface. The pads 12, 14 and 16 act to keep the middle finger 24 from bending as much as it normally would in gripping the object 86. As such, the apparatus 10 creates laxity in the connection between the tendon of the finger, the muscle of the forearm pulling on the tendon of the finger, and the tendon in the elbow attached to the muscle of the forearm. By preventing the middle finger 24 from fully bending, the aforementioned tendon-muscle-tendon unit is not completely tight and enjoys some degree of looseness. The apparatus 10 thus causes a greater gripping circumference of the middle finger 24. The pads 12, 14 and 16 do not interfere with the bending of the interphalangeal 46 and 48 or metacarpalphalangeal joints of the hand of the user 18 yet engage the finger 24 in a manner so as to be precluded from twisting or slipping around the finger 24 and out of position.

Impact forces on the object 86 are transferred through the finger 24 and the tendon-muscle-tendon unit. These forces act to cause tendinitis and are more quickly and strongly propagated if the tendon-muscle-tendon unit is tight in much the same way a wave is more strongly and quickly propagated along a tighter rather and looser wire. As the apparatus 10 causes the tendon-muscle-tendon unit to be looser than normal, impact forces on the object 86 are of a lower magnitude on and speed through the tendon-muscle-tendon unit. As such, the resulting pull forces on the elbow of the user 18 are reduced thus preventing or reducing tendinitis. The apparatus 10 thus acts to decrease the strength of the shock wave imparted and slows the propagation of the diminished shock wave so that it dissipates and exerts decreased pull upon the tendon at the elbow.

Additionally, as the tendon-muscle-tendon unit has a degree of laxity imparted thereto, there is little chance of exceeding the tensile strength of the tendon even if a higher impact than usual is transmitted to the object 86 such as when an off-center or high velocity shot is hit while playing tennis. Also, as the pads 12, 14 and 16 have relatively large thicknesses 72, 74 and 76 and are made of a resilient shock absorbing material they further act to dampen impact forces imparted onto the object 86. Here, the pads 12, 14 and 16 directly absorb some of the kinetic energy transmitted from the object 86 at the grip interface.

As the pads 12, 14 and 16 are positioned between the finger 24 of the user and the object 86 their proper positioning may be easily ascertained by the user. Continued use of the object 86 will not cause the apparatus 10 to lose any amount of resiliency and, as such, the apparatus 10 does not need to be readjusted or tightened during use. The apparatus 10 may be used in conjunction with other types of elbow braces and pads in addition to being used with specially designed grips. Although described as being used in relation to golf and tennis, the apparatus 10 may be used in relation to any activity in which a user 18 grasps an object 86. For example, the apparatus 10 can be used in relation to hockey, baseball, softball, fishing, rope climbing, wind surfing, rowing, bicycling and snowmobiling. Further activities in which the apparatus 10 may be used include housecleaning, the use of hand tools, computer use, use of a chainsaw, lawnmower and garden tool use, use of musical instruments, wheel barrow use, crane operation, window washing, holding of retractors during surgery, plumbing work, hair dryer use, exercise equipment use, handicap fall bar use, wheelchair use, physical therapy equipment use, painting and truck loading. The apparatus 10 can be used in any application that involves repetitive motion or shock to the hand of the user 18.

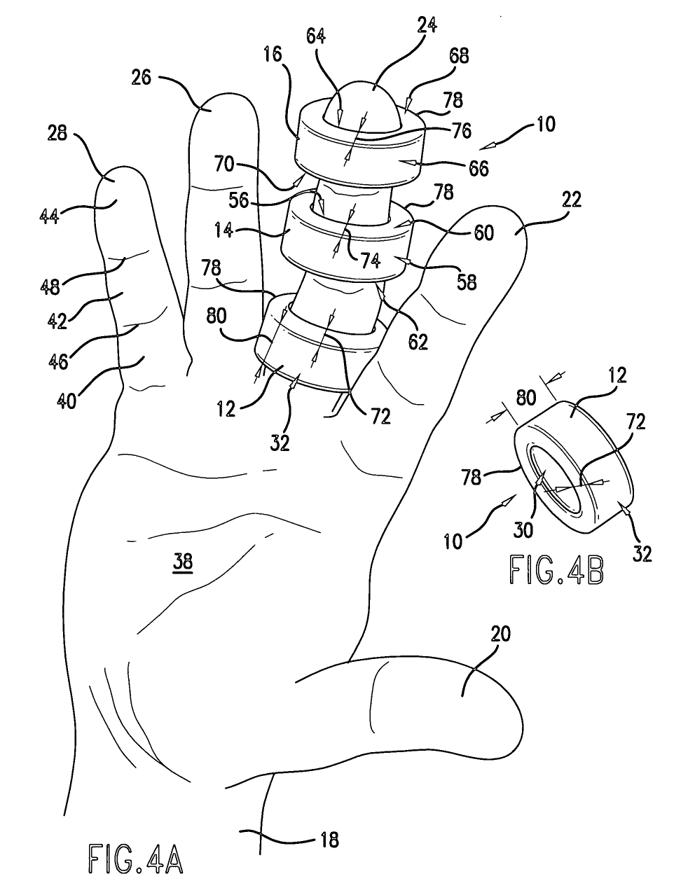

The apparatus 10 can be configured differently in accordance with other exemplary embodiments of the present invention. FIG. 4A shows the apparatus 10 having pads 12, 14 and 16 that are configured as rings that extend completely around the middle finger 24. First pad 12 is shown in FIG. 4B as being a ring 78 that has a constant thickness 72 about its perimeter. Further, the width 80 of the ring 78 is constant about its axis. Both the thickness 72 and width 80 may be up to ten millimeters in various embodiments. Although described as having a constant thickness 72 and width 80, it is to be understood that the thickness 72 and width 80 may be varied in other exemplary embodiments. Providing the apparatus with pads 12, 14 and 16 in the shape of a ring may be advantageous in that a loss of contact area does not occur even if the pads 12, 14 and 16 twist or twirl around the middle finger 24 during use. Further, the increased amount of resilient material present from having the pads 12, 14 and 16 extend around the entire middle finger 24 may act to absorb a greater amount of shock from impact forces than if the pads 12, 14 and 16 were located on only one side of the finger 24. As with other embodiments, the pads may be supplied in any number and located on various fingers of the user 18.

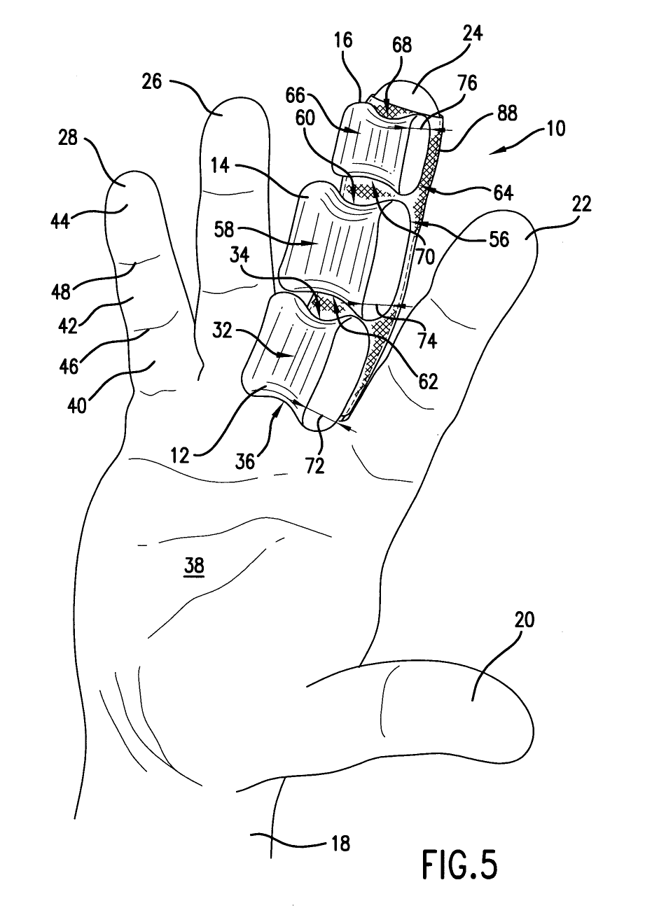

FIG. 5 shows another exemplary embodiment of the apparatus 10. Here, three pads 12, 14 and 16 are present and are incorporated into a sheath 88. The sheath 88 fits over the middle finger 24 and can be positioned so that the pads 12, 14 and 16 are located on the palmar side of the finger 24. The sheath 88 may be made of an elastic material that fits snugly onto finger 24. The pads 12, 14 and 16 may be attached to the sheath 88 in a variety of manners. For example, the pads 12, 14 and 16 can be attached through adhesion, hook and look type fasteners, mechanical fasteners, placed into pockets of the sheath 88 or may be integrally formed with the sheath 88. The pads 12, 14 and 16 may be discontinuous in order to more easily allow the finger 24 to bend. Although shown as having three pads 12, 14 and 16, any number of pads may be used in accordance with other embodiments. Further, the sheath 88 can be configured in a variety of manners. For example, the sheath 88 may be provided in any manner such as that disclosed in U.S. Pat. No. 6,839,905 to Bruder et al., the entire contents of which are incorporated by reference herein in their entirety for all purposes.

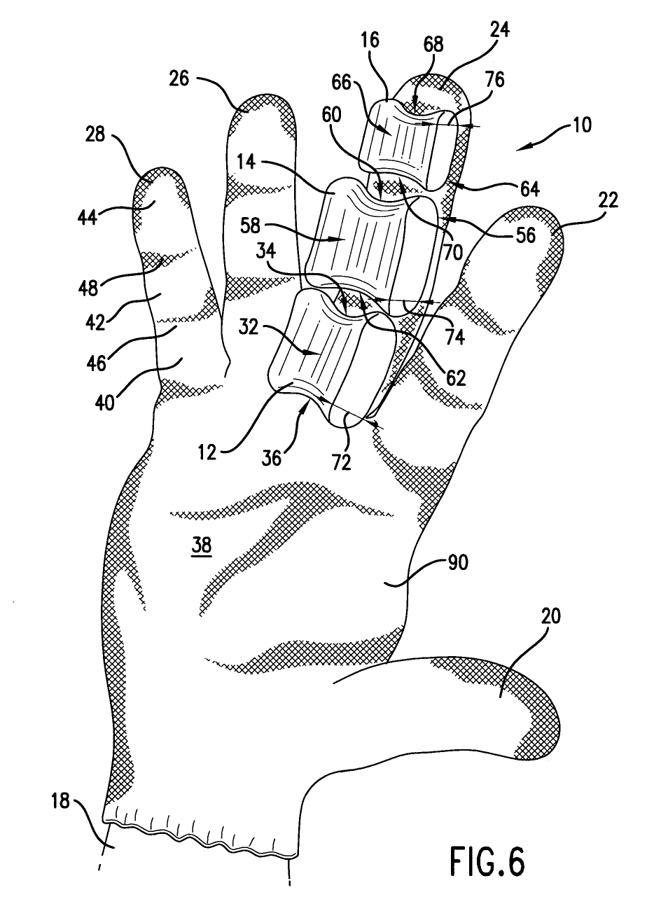

Yet another embodiment of the apparatus 10 is shown in FIG. 6. The apparatus 10 is shown as having three pads 12, 14 and 16 that are incorporated into a glove 90 that acts to retain the pads 12, 14 and 16 onto the finger 24 of the user 18. The pads 12, 14 and 16 can be carried by the glove 90 through adhesion, hook and look type fasteners, mechanical fasteners, pockets of the sheath 90 or integrally formed with the glove 90. The pads may be placed on any number of the fingers 20, 22, 24, 26 or 28 of the glove 90. As with the previously described embodiments, the pads 12, 14 and 16 may be positioned so as to be disposed between the fingers of the user 18 and the object 86 grasped by the user 18 so that the tendon-muscle-tendon unit of the user 18 retains some degree of slackness. The glove 90 may be provided in a variety of manners. For example, the glove 90 may be provided as that described in U.S. Pat. No. 4,159,116 to Geist and as that in U.S. Pat. No. 3,922,724 to Shockovsky. The entire contents of U.S. Pat. Nos. 4,159,116 and 3,922,724 are incorporated by reference herein in their entirety for all purposes.

While the present invention has been described in connection with certain preferred embodiments, it is to be understood that the subject matter encompassed by way of the present invention is not to be limited to those specific embodiments. On the contrary, it is intended for the subject matter of the invention to include all alternatives, modifications and equivalents as can be included within the spirit and scope of the following claims.

What is claimed:

1. An apparatus for preventing the development or exacerbation of tendinitis, comprising:
a first pad made of a resilient material and configured for being carried by a digit of the user, said first pad having a palmar face and an oppositely disposed dorsal face, said first pad having a first end and an oppositely disposed second end, wherein at least one of said first and second ends has a concave shape to allow for some degree of bending of a digit of the user.

2. The apparatus as in claim 1, further comprising a retaining member configured for retaining said first pad to a digit of the user.

3. The apparatus as in claim 2, wherein said retaining member contacts said dorsal face of said first pad.

4. The apparatus as in claim 2, wherein said retaining member is selected from the group consisting of a hook and loop type fastener, gauze, athletic tape, an elastic member, a finger cot, and a glove.

5. The apparatus as in claim 1, wherein said palmar face has a concave shape configured for receiving a phalange of the user.

6. The apparatus as in claim 1, wherein both said first end and said second end have a concave shape.

7. The apparatus as in claim 1, further comprising a second pad made of a resilient material and configured for being carried by a digit of the user, said second pad having a palmar face and an oppositely disposed dorsal face, said second pad having a first end and an oppositely disposed second end, wherein at least one of said first and second ends has a concave shape to allow for some degree of bending of a digit of the user.

8. The apparatus as in claim 7, further comprising a third pad made of a resilient material and configured for being carried by a digit of the user, said third pad having a palmar face and an oppositely disposed dorsal face, said third pad having a first end and an oppositely disposed second end, wherein at least one of said first and second ends has a concave shape to allow for some degree of bending of a digit of the user.

9. An apparatus for preventing the development or exacerbation of tendinitis, comprising:
a first pad made of a resilient material and configured for being carried by a digit of the user, said first pad having a palmar face and an oppositely disposed dorsal face that define a thickness of said first pad therebetween, wherein said thickness of said first pad is selected so as to prevent the digit of the user from fully bending.

10. The apparatus as in claim 9, wherein the thickness of said first pad is at least nine millimeters.

11. The apparatus as in claim 9, further comprising a retaining member configured for retaining said first pad to the digit of the user.

12. The apparatus as in claim 11, wherein said retaining member is selected from the group consisting of a hook and loop type fastener, gauze, athletic tape, an elastic member, a finger cot, and a glove.

13. The apparatus as in claim 9, wherein said palmar face has a concave shape configured for receiving a phalange of the user, wherein said first pad has a first end and an oppositely disposed second end, and wherein at least one of said first and second ends has a concave shape.

14. The apparatus as in claim 9, wherein said first pad is a ring that has a uniform width and thickness.

15. The apparatus as in claim 9, further comprising a second pad made of a resilient material and configured for being carried by a digit of the user, said second pad having a palmar face and an oppositely disposed dorsal face that define a thickness of said second pad therebetween, wherein said thickness of said second pad is selected so as to prevent the digit of the user from fully bending.

16. The apparatus as in claim 15, further comprising a third pad made of a resilient material and configured for being carried by a digit of the user, said third pad having a palmar face and an oppositely disposed dorsal face that define a thickness of said third pad therebetween, wherein said thickness of said third pad is selected so as to prevent the digit of the user from fully bending.

17. A method of preventing the development or exacerbation of tendinitis, comprising the steps of:
providing a first pad made of a resilient material and configured for being carried by a digit of the user, said first pad having a palmar face and an oppositely disposed dorsal face that define a thickness of said first pad therebetween, wherein said thickness of said first pad is selected so as to prevent the digit of the user from fully bending; and
retaining said first pad onto the digit of the user such that said first pad is positioned on the palmar side of the digit.

18. The method as in claim 17, further comprising the steps of:
grasping an object with a hand onto which said first pad is retained such that the digit onto which said first pad is retained curls onto the object without tightly grasping the object; and
absorbing impact forces imparted onto the object by way of resiliency of said first pad and by way of slackness of a muscle pulling on the digit.

19. The method as in claim 17, further comprising the steps of:
providing a second pad made of a resilient material and configured for being carried by a digit of the user, said second pad having a palmar face and an oppositely disposed dorsal face that define a thickness of said second pad therebetween, wherein said thickness of said second pad is selected so as to prevent the digit of the user from fully bending; and
retaining said second pad onto the digit of the user such that said second pad is positioned on the palmar side of the digit.

20. The method as in claim 19, further comprising the steps of:
providing a third pad made of a resilient material and configured for being carried by a digit of the user, said third pad having a palmar face and an oppositely disposed dorsal face that define a thickness of said third pad therebetween, wherein said thickness of said third pad is selected so as to prevent the digit of the user from fully bending; and
retaining said third pad onto the digit of the user such that said third pad is positioned on the palmar side of the digit, wherein said first, said second and said third pads are retained on the same digit of the user.
